(19) United States
(12) Patent Application Publication
(10) Pub. No.: US 2006/0147495 A1
(43) Pub. Date: Jul. 6, 2006

(54) Amino acid-based nutritional formulation

(76) Inventors: Ian Sullivan, Merseyside (GB); Johanna Hill, Liverpool (GB)

Correspondence Address: WOODARD, EMHARDT, MORIARTY, MCNETT & HENRY LLP, 111 MONUMENT CIRCLE, SUITE 3700, INDIANAPOLIS, IN, 46204-5137, US

(21) Appl. No.: 11/026,762

(22) Filed: Dec. 30, 2004

(51) Int. Cl. A61K 31/734; A61K 31/717; A61K 31/198; A61K 47/00

(52) U.S. Cl. 424/439; 514/567; 514/57; 514/54

(57) ABSTRACT

A ready to feed nutritional formulation for consumption by persons with an inborn error of metabolism, the formulation comprising: a homogenised blend water, calcium and amino acid moieties in a profile appropriate to the inborn error, said amino acid moieties being selected from the group consisting of free amino acids, protein hydrolysates and derivatives of such acids and hydrolysates characterised in that (i) the formulation contains at least 5% w/v of said amino acid moieties, at least some of which are in suspension by virtue of being present in the formulation in amounts above their solubility limit, (iii) the calcium is present in an amount of at least 0.1% by weight, and (iii) the formulation includes a gelatinising stabiliser to maintain insoluble solids in suspension.

### FIELD OF THE INVENTION

The invention relates to a nutritional, amino acid-based formulation, and more particularly to such a formulation of the “ready-to-feed” type intended for consumption by persons with an inborn error of metabolism.

### BACKGROUND OF THE INVENTION

The dietary management of a number of clinical conditions requires the provision of nutritional nitrogen in the form of individual amino acids or protein hydrolysates rather than as whole protein. For example, many diseases resulting from inborn errors of metabolism exhibit dysfunctional amino acid metabolism. Phenylketonuria, in which the enzyme for Phenylalanine (Phe) metabolism is impaired or absent, causes chronic, toxic high levels of plasma and tissue Phe. Disease management lowers blood and tissue Phe levels by providing free amino acids (excepting Phe) as a substitute for normal protein (which contains Phe) in the diet.

Amino acid formulas for the dietary management of such conditions are generally provided as dry powders for re-constitution, as these provide product stability over long periods and so contribute to shelf-life. One example of such a dry powder product is Maxamum (ex SHS) which is a nutritionally complete product used as a protein substitute in a managed diet regime. The diet therefore consists mostly or entirely of liquid meals which must be mixed from a packet before use, and is therefore inconvenient to personal lifestyle for daily and long-term use. The high volumes of product required to deliver dietary levels of nitrogen also significantly impact user convenience. These restrictions of current product formats make it very difficult for patients to integrate dietary management into their everyday life, such as adults at work or children at school. There is the further disadvantage that the reconstituted product is not storage stable and must be consumed relatively quickly (i.e., within 24 hours) so it is not possible to make up “volumes” for later use.

Several innovations have emerged to increase user convenience of amino acid formulas, making the diets easier to use for patients and so improving quality of life, compliance and clinical effectiveness. One development has been the provision of ready-to-feed (RTF) nutritional amino acid formulas, such formulas being manufactured and supplied as storage stable liquids. For example, Elemental E028 Extra Liquid® (ex SHS International) is a liquid RTF amino acid formula used as the sole source of nutrition for the dietary management of several conditions intolerant of whole protein, such as Crohn's disease, short bowel syndrome, intractable malabsorption and radiation enteritis. Elemental E028 contains 3% w/v amino acids and has a shelf-life of 9 months. Although this product format has the convenience of being ready-to-feed, the relatively low concentration of amino acids/nutrients means that relatively large volumes still have to be carried around, restricting positive impact on lifestyle.

A major problem with current liquid elemental compositions is product stability, in that they are inherently prone to phase separation on standing. This problem is exacerbated by higher concentrations of amino acids. Higher concentrations would be beneficial in RTF formulas because of reduction of product volume to deliver a specific amount of nutrition, thereby improving user convenience. However, the extremely bad taste of high concentration amino acids limits the volume restriction that can practically be included in RTF formulas. Use of existing RTF product formats to provide high levels of amino acids in a small volume is therefore not an option due to both the extreme bitterness of the preparations and the significant instability of the products.

Several inventions exist addressing these problems in high volume, low concentration RTF formulas, but do not address the problems of low volume, high concentration RTF formulas.

U.S. Pat. No. 6,017,550 teaches the use of dietary fibres to improve the stability of liquid RTF amino acid formulas, but does not address the problems of low volume, high concentration formulas.

U.S. Pat. No. 6,436,464 teaches the use of an emulsifying system comprising an octenyl succinic anhydride modified starch and an acetylated monoglyceride emulsifier to improve the stability of liquid RTF amino acid formulas, but does not address the problems of low volume, high concentration formulas.

CA-A-2 163 379 teaches specific liquid RTF formulations containing amino acids, protein hydrolysate and whole protein in defined ratios which have improved emulsion and storage stability, but does not address the problems of low volume, high concentration formulas.

U.S. Pat. No. 5,985,339 teaches liquid RTF amino acid formulas which are nutritionally complete and are stable when refrigerated, but does not address the problems of low volume, high concentration formulas.

None of the inventions comprising the state-of-the-art therefore meet the requirements of optimised user convenience outlined above, i.e., provision of high concentration, low volume RTF amino acid formulas.

### SUMMARY OF THE INVENTION

The invention provides a ready to feed nutritional formulation for consumption as a protein substitute by persons with an inborn error of metabolism, the formulation comprising:

a homogenised blend of water, calcium and amino acid moieties in a profile appropriate to the inborn error, said amino acid moieties being selected from the group consisting of free amino acids, protein hydrolysates and derivatives of such acids and hydrolysates

characterised in that

(i) the formulation contains at least 5% weight by volume (% w/v) of said amino acid moieties, at least some of which are in suspension by virtue of being present in the formulation in amounts above their solubility limit, and

(iii) the formulation includes a gelatinising stabiliser to maintain insoluble solids in suspension.

The invention thus provides a ready to feed formulation intended to be used as a protein substitute (without necessarily being the sole source of nutrition) in a managed diet for a person having an inborn error of metabolism. Compositions in accordance with the invention may be formulated for a number of inborn error of metabolism conditions, e.g. phenylketnouria (PKU), homocystinuria (HCU), Maple Syrup Urine Disease (MSUD), tyrosinaemia, propionic acidaemia, methylmalonic acidaemia, isovaleric acidaemia, urea cycle disorders and glutaric aciduria.

### DESCRIPTION OF PREFERRED EMBODIMENTS

A composition in accordance with the invention will, be formulated having regard to a particular inborn error of metabolism condition (e.g. PKU, HCU or MSUD). However irrespective of the particular condition, the composition will contain at least 5% w/v of amino acid moieties as provided by free amino acids, derivatives thereof (e.g. salts), protein hydrolysates or derivatives thereof. Compositions in accordance with the invention may however be formulated with higher amounts of amino acids moieties, e.g. at least 8% w/v. Particular examples of compositions in accordance with the invention may contain more than 10% w/v of the amino acid moieties or even more than 20% w/v. Generally the total amount of amino acid moieties will not exceed 55% w/v of the composition. Additionally the composition preferably contains at least 0.05% w/v (more preferably at least 0.1% w/v) of calcium ions. Such high amounts of calcium are important because the amino acid moieties act as a substitute for protein that would normally be derived from milk which provides a relatively high amount of calcium, which must be made up if the amino acid moieties provide nitrogen instead.

The relatively high amounts of amino acid moieties and calcium mean that the formulation is relatively concentrated as compared to prior art ready-to-feed formulations, which are not intended for use as protein substitutes in persons with an inborn error of metabolism. Thus the disadvantages of the high volume liquid products (namely large volumes required to provide the required amino acid intake) are avoided.

With such high concentrations, it will be the case that at least one of the amino acid moieties is present at a level above its solubility limit. Generally this amino acid will be tyrosine (except in the case of products for tyrosinaemia). Alternatively or additionally the amino acid moiety that is present above its solubility limit will be cystine. Thus, for example, a formulation intended for consumption by a persons having PKU as an inborn error of metabolism will be devoid of phenylalanine but will contain a high amount of tyrosine.

The gelatinising stabiliser produces a gel stabilised system. Such a stabiliser serves two functions. The first is to maintain insoluble solids in suspension so as to provide a storage stable composition that does not sediment for prolonged periods of standing. The suspended solids will include some of the amino acid moieties (i.e. those that are present above their solubility limit) and will generally also include minerals present in the composition (see infra). The second function of the stabiliser is to provide a product having a good palatability, this being a combination of mouthfeel and taste both of which are improved by the stabilisation system.

A gelatinising stabiliser is one that stabilises via its gel structure and provides an increase in viscosity such as to allow the suspended solids to be maintained in suspension. A gel stabilised system can be described as a colloidal suspension comprised of a continuous phase (water) and a dispersed phase of the gelling agent. Water and other components of the formulation are held in a three dimensional network of the gelling agent(s).

Generally the gelatinising stabiliser will provide the formulation with a viscosity of at least 8 mPas@1000/s (as measured using a CSL 2 Rheometer Carrimed@20° C., cone plate spindle@ 1000/s). Preferably the viscosity is 8 to 30 mPas@ 1000/s (measured as defined above).

Preferably also the gelatinising stabiliser is heat-stable and freeze/thaw-stable.

The gelatinising stabiliser preferably comprises a polysaccharide mixture. Preferably the mixture comprises a soluble hydrocolloid component and an insoluble hydrocolloid component. Preferably the soluble hydrocolloid component comprises guar and/or xanthan.gum (preferably a mixture of both gums). Preferably the insoluble hydrocolloid component comprises modified cellulose. Preferably the modified cellulose is microcrystalline cellulose. (Typically the weight ratio of microcrystalline cellulose: guar gum: xanthan gum will be 5:2-3:1.5-2.5. A particularly suitable ratio is about 5:2.6:2.05.

Generally the total amount of the gelatinising stabiliser in the formulation will be at least 0.2% w/v, preferably 2.5 to 5% w/v, e.g. about 0.39% w/v.

The microcrystalline cellulose may, for example, be Avicel as available from FMC Corporation. The soluble hydrocolloid component may be the mixture of xanthan and guar gum available as Meyprogen MS-604 as available from Rhodia Ltd.

The amino acid moieties are preferably either free amino acids or derivatives thereof (e.g. salts, esters or acyl derivatives) although we do not preclude the use of protein hydrolysates which might for example have a molecular weight up to 5 kDa although size is not particularly important in the dietary management of an inborn error of metabolism since the amino acid profile is the most important characteristic.

The formulation preferably comprises at least amino acid moieties corresponding to the “essential” amino acids, i.e. histidine, isoleucine, leucine, lysine, methionine (and/or cysteine), phenylalanine (and/or tyrosine), threonine, tryptophan and valine.

The formulation may also include one or more of the following amino acids, namely: alanine, arginine, asparagine, aspartic acid, cysteine (if not present as one of the essential amino acids), glutamic acid, glutamine, glycine, proline, serine, tyrosine and valene.

Methionine present in the composition is preferably provided by a methionine derivative having a solubility of at least 5.0 g/100 ml (in deionised water at 20° C.). Most preferably methionine is provided by acetyl methionine which provides a distinct taste advantage. Lysine is preferably provided by a lysine salt with a solubility of at least 65 g/100 ml (in deionised water at 20° C.). A suitable lysine salt is lysine acetate.

It is preferred that, for stability and taste reasons, the formulation does not contain glutamine or glutamic acid.

Formulations in accordance with the invention preferably contain 5% to 55% w/v of amino acid moieties (based on the total weight of the formulation). Preferably the formulation includes at least some of the following amino acid moieties listed in Table A in the ranges indicated, it being appreciated that certain in born errors of metabolism will dictate the presence or absence of particular amino acid moieties. Generally the amounts at the lower end of the range will be employed in formulations containing a total of 5% w/v of amino acid moieties whereas amounts at the upper end of the range will be used for formulations containing a total of 55% w/v of the amino acid moieties. Obviously it is possible proportionately to calculate corresponding amounts for each amino acid when the total amount of amino acids is intermediate the preferred limits of 5% and 55%.

TABLE A

| | Minimum | Maximum |
|---|---|---|
| ALANINE | 0.20 | 3.24 |
| ARGININE | 0.35 | 5.71 |
| ASPARTIC ACID | 0.30 | 4.74 |
| CYSTINE | 0.13 | 2.12 |
| GLUTAMIC ACID | 0.00 | 0.29 |
| GLYCINE | 0.25 | 3.88 |
| HISTIDINE | 0.20 | 3.23 |
| ISO-LEUCINE | 0.00 | 3.66 |
| LEUCINE | 0.00 | 6.26 |
| LYS/GLUTAMATE | 0.00 | 10.56 |
| PHENYLALANINE | 0.00 | 3.85 |
| PROLINE | 0.39 | 6.16 |
| SERINE | 0.24 | 3.78 |
| THREONINE | 0.27 | 4.28 |
| TRYPTOPHAN | 0.11 | 1.69 |
| TYROSINE | 0.00 | 5.54 |
| VALINE | 0.00 | 4.02 |
| GLUTAMINE | 0.00 | 0.62 |
| LYSINE ACETATE | 0.00 | 8.33 |
| TAURINE | 0.01 | 0.24 |
| METHIONINE | 0.00 | 1.38 |
| CARNITINE | 0.00 | 0.03 |
| N-Acetyl L- | 0.00 | 1.29 |
| Methionine | | |

The following Table B gives exemplary amino acid profiles for formulations in accordance with the inventions intended for HCU, MSUD, Tyrosinaemia and PKU patients.

TABLE B

| | HCU | | MSUD | | Tyrosenemia | | PKU | |
|---|---|---|---|---|---|---|---|---|
| | AA concen- | | AA concen- | | AA concen- | | AA concen- | |
| | tration | | tration | | tration | | tration | |
| | 5% | 55% | 5% | 55% | 5% | 55% | 5% | 55% |
| ALANINE | 0.20 | 2.24 | 0.29 | 3.24 | 0.20 | 2.15 | 0.20 | 2.19 |
| ARGININE | 0.35 | 3.90 | 0.52 | 5.71 | 0.37 | 4.06 | 0.38 | 4.15 |
| ASPARTIC ACID | 0.30 | 3.26 | 0.43 | 4.74 | 0.34 | 3.78 | 0.35 | 3.86 |
| CYSTINE | 0.13 | 1.45 | 0.19 | 2.12 | 0.14 | 1.50 | 0.16 | 1.72 |
| GLUTAMIC ACID | 0.03 | 0.29 | 0.00 | 0.00 | 0.03 | 0.28 | 0.00 | 0.00 |
| GLYCINE | 0.32 | 3.47 | 0.25 | 2.73 | 0.34 | 3.79 | 0.35 | 3.88 |
| HISTIDINE | 0.20 | 2.23 | 0.29 | 3.23 | 0.21 | 2.34 | 0.21 | 2.35 |
| ISO-LEUCINE | 0.32 | 3.47 | 0.00 | 0.00 | 0.33 | 3.58 | 0.33 | 3.66 |
| LEUCINE | 0.54 | 5.99 | 0.00 | 0.00 | 0.56 | 6.13 | 0.57 | 6.26 |
| LYS/GLUTAMATE | 0.83 | 9.13 | 0.00 | 0.00 | 0.96 | 10.56 | 0.00 | 0.00 |
| PHENYLALANINE | 0.24 | 2.63 | 0.35 | 3.85 | 0.00 | 0.00 | 0.00 | 0.00 |
| PROLINE | 0.39 | 4.24 | 0.56 | 6.16 | 0.40 | 4.35 | 0.40 | 4.43 |
| SERINE | 0.24 | 2.61 | 0.34 | 3.78 | 0.24 | 2.68 | 0.25 | 2.74 |
| THREONINE | 0.27 | 2.92 | 0.39 | 4.28 | 0.27 | 3.00 | 0.28 | 3.07 |
| TRYPTOPHAN | 0.11 | 1.17 | 0.15 | 1.69 | 0.11 | 1.20 | 0.11 | 1.22 |
| TYROSINE | 0.24 | 2.63 | 0.35 | 3.85 | 0.00 | 0.00 | 0.50 | 5.54 |
| VALINE | 0.35 | 3.81 | 0.00 | 0.00 | 0.36 | 3.92 | 0.37 | 4.02 |
| GLUTAMINE | 0.04 | 0.44 | 0.00 | 0.00 | 0.06 | 0.62 | 0.00 | 0.00 |
| LYSINE ACETATE | 0.00 | 0.00 | 0.76 | 8.33 | 0.00 | 0.00 | 0.62 | 6.77 |
| TAURINE | 0.01 | 0.10 | 0.02 | 0.22 | 0.02 | 0.21 | 0.02 | 0.24 |
| METHIONINE | 0.00 | 0.00 | 0.13 | 1.38 | 0.09 | 0.96 | 0.00 | 0.00 |
| CARNITINE | 0.00 | 0.00 | 0.002 | 0.03 | 0.00 | 0.03 | 0.00 | 0.03 |
| N-Acetyl L- | 0.00 | 0.00 | 0.00 | 0.00 | 0.00 | 0.00 | 0.12 | 1.29 |
| Methionine | | | | | | | | |

As indicated above, formulations in accordance with the invention preferably contain calcium, most preferably in an amount of at least 0.05% w/v, more preferably at least 0.1% w/v.

Preferably the calcium is provided by a calcium salt having a solubility in deionised water at 12° C. of at least 0.1 g/100 ml. Preferably this solubility is at least 0.5 g/100 ml, and more preferably at least 1 g/100 ml. A particularly preferred calcium salt for use in producing a formulation in accordance with the invention is calcium glycerophosphate, which has a solubility (under the defined conditions) of about 2 g/100 ml.

Minerals additional to calcium may be present in the formulation, e.g. one or more of sodium, potassium, chloride, phosphorous and magnesium. Sodium is preferably provided by a salt thereof having a solubility (at 25° C. in deionised water) of at least 35 g/100 ml (e.g. sodium chloride). Potassium is preferably provided by a salt having a solubility of at least 35.5 g/100 ml (at 25° C. in deionised water), e.g. tripotassium citrate or dipotassium hydrogen phosphate.

The formulation may contain trace elements e.g. one or more of iron, zinc, iodine, manganese, copper, molybdenum, selenium, chromium and fluoride.

The formulation may contain vitamins selected from Vitamin A, Vitamin E, L-ascorbic acid, thiamin, riboflavin, pyridoxine, niacin, pantothenic acid, myoinositol, choline, Vitamin D3, cyanocobalamin, folacin, d-biotin and Vitamin K1.

The formulation may also incorporate a fat. The total amount of fat present in the composition may for example be up to 2% w/v. Examples of suitable fats include high oleic sunflower oil, canola oil and walnut oil. Preferably a blend of all three of these fats are used. Additional components that may be present in the formulation include carbohydrate and fibre. The carbohydrate may for example be provided by Sugar & Dried Glucose Syrup (e.g. DE21 Lane Eynon) whereas the fibre may be provided by Xanthan, MCC and Guar Gum.

Preferably the formulation is nutritionally complete.

A flavouring is preferably included in the formulation at a level of at least 0.65 g/100 ml.

Formulations in accordance with the invention may be prepared by initially mixing the various components of the formulation (in the required amounts) with water to produce a slurry. Subsequently this composition may be subjected to a UHT sterilisation treatment (e.g. at about 144° C.). A suitable sterilisation technique is direct steam injection. The composition may then be flash cooled, e.g. to 80°-85° C. and then further cooled to a temperature, e.g. about 70° C., at which the composition may be homogenised. It is preferred that a two stage homogenisation procedure is used, the first stage using a first homogenisation pressure of about 150-170 bar (e.g. 160 bar) followed by a second lower pressure homogenisation of about 30-50 bar (e.g. about 40 bar). The resulting formulation may then be cooled/chilled to 10-15° temperature and then packaged.

### EXAMPLE

A formulation intended for consumption by PKU patients was prepared from water and for admixtures A-D as listed in Table 1 below.

| | | Net Weight |
|---|---|---|
| | | (grams) per 1000 |
| Compo- | | litres of PKU |
| sition | Material | formulation |
| | WATER | 810000.00 |
| A | AVICEL GP 3252 | 2000.00 |
| B | HIGH OLEIC SUNFLOWER OIL | 9940.00 |
| | CANOLA OIL | 7540.00 |
| | WALNUT OIL | 920.00 |
| | DIMODAN HR | 900.00 |
| | ADMUL DATEM 1117 | 900.00 |
| C | LYSINE ACETATE | 9839.93 |
| | LEUCINE | 9110.17 |
| | TYROSINE | 8059.84 |
| | PROLINE | 6449.93 |
| | ARGININE | 6039.94 |
| | VALINE | 5839.69 |
| | GLYCINE | 5640.23 |
| | ASPARTIC ACID | 5619.65 |
| | ISO-LEUCINE | 5319.67 |
| | THREONINE | 4470.39 |
| | HISTIDINE | 3420.07 |
| | ALANINE | 3189.75 |
| | CYSTINE | 2500.35 |
| | ACETYL METHIONINE | 1870.31 |
| | TRYPTOPHAN | 1780.08 |
| | FLAVOURING | 7400.00 |
| | SERINE | 3990.13 |
| | TAURINE | 355.31 |
| | CARNITINE | 47.50 |
| | PULVERISED SUGAR | 1497.06 |
| | CITRIC ACID | 4000.00 |
| | VITAMIN A ACETATE | 20.13 |
| | DRY VITAMIN E 50% | 30.53 |
| | ASCORBIC ACID | 1013.46 |
| | THIAMIN HYDROCHLORIDE | 3.71 |
| | RIBOFLAVIN | 2.75 |
| | PYRIDOXINE HYDROCHLORIDE | 5.07 |
| | NICOTINAMIDE | 26.59 |
| | CALCIUM D-PANTOTHENATE | 11.22 |
| | VITAMIN D3 | 7.17 |
| | VITAMIN B12 0.1% | 8.50 |
| | FOLIC ACID 10% | 10.20 |
| | D-BIOTIN 1% | 27.78 |
| | DRY VITAMIN K1 5% | 3.71 |
| | POTASSIUM IODIDE | 0.31 |
| | INOSITOL | 167.55 |
| | CHOLINE BITARTRATE | 1316.48 |
| | MALTODEXTRIN | 1554.85 |
| | SUNETT | 400.00 |
| D | CALCIUM GLYCEROPHOSPHATE | 9040.00 |
| | IRON SULPHATE 7H20 | 201.10 |
| | ZINC SULPHATE 7H20 | 102.17 |
| | MANGANESE SULPHATE 4H20 | 14.33 |
| | SODIUM MOLYBDATE 2H20 | 0.46 |
| | SODIUM HYDROGEN SELENITE | 0.16 |
| | CHROMIUM SULPHATE SOLUTION | 34.34 |
| | MALTODEXTRIN | 2918.31 |
| | COPPER SULPHATE 5H20 | 9.12 |
| | MAGNESIUM ACETATE | 2990.00 |
| | ANHYDROUS. | |
| | TRI-POTASSIUM CITRATE 1H20 | 2850.00 |
| | MEYPROGEN MS-604 | 2000.00 |
| | SODIUM CHLORIDE | 1570.00 |
| | TRI-SODIUM CITRATE | 1140.00 |
| | DIHYDRATE | |
| | DI-POTASSIUM HYDROGEN | 220.00 |
| | PHOSPHATE ANHYDROUS | |
| | GRANULATED SUGAR | 45000.00 |

The formulation was obtained by the following procedure:
(i) Composition A was thoroughly mixed with the water.
(ii) Composition B was pre-heated to 75-80° C. and then thoroughly mixed with the product of step (i).
(iii) Composition D was then added to the product of step (ii) and thoroughly mixed.
(iv) Composition C was then added with thorough mixing.

The product from step (iv) was heated to about 80° C. and then subjected to UHT sterilisation by direct steam injection for four seconds.

After cooling to 70° C., the product was then subjected to a two stage homogenisation procedure, the first stage being at 160 bar and the second stage being at 40 bar. Both homogenisation stages were affected at 70° C. Finally the product was cooled to 20-30° C. and then chilled to 10-15° C.

The resultant product had a “creamy” mouthfeel and had a much smoother texture as compared to that of formulations obtained by simply hand-mixing the existing powdered equivalents (e.g. PKU Express (Vitaflo), Maxamum (SHS), Phenex (Ross), Lofenelac (Mead Johnson), PKU 1,2 (Milupa).

1. A ready to feed nutritional formulation for consumption by persons with an inborn error of metabolism, the formulation comprising:
a homogenised blend of water and amino acid moieties in a profile appropriate to the inborn error, said amino acid moieties being selected from the group consisting of free amino acids, protein hydrolysates and derivatives of such acids and hydrolysates
characterised in that
(i) the formulation contains at least 5% w/v of said amino acid moieties, at least some of which are in suspension by virtue of being present in the formulation in amounts above their solubility limit,
(ii) the formulation includes a gelatinising stabiliser to maintain insoluble solids in suspension.

2. A formulation as claimed in claim 1 wherein the amino acid moieties are provided by free amino acids or derivatives thereof.

3. A formulation as claimed in claim 1 wherein the amino acid moiety that is present above its solubility level is tyrosine.

4. A formulation as claimed in claim 1 containing calcium in an amount of at least 0.05% w/v.

5. A formulation as claimed in claim 4 wherein the amount of calcium is at least 0.01% w/v.

6. A formulation as claimed in claim 4 wherein the calcium is provided by a calcium salt having a solubility in deionised water at 12° C. of at least 0.1 g/100 ml, and

7. A formulation as claimed in claim 6 wherein the calcium is provided by calcium glycerophosphate.

8. A formulation as claimed in claim 1 wherein the gelatinising stabiliser comprises a polysaccharide mixture.

9. A formulation as claimed in claim 8 wherein the gelatinising stabiliser comprises a soluble hydrocolloid component and an insoluble hydrocolloid component.

10. A formulation as claimed in claim 9 wherein the soluble hydrocolloid component is selected from the group consisting of guar gum, xanthan gum and mixtures thereof.

11. A formulation as claimed in claim 10 wherein the soluble hydrocolloid component comprises a mixture of guar gum and xanthan gum.

12. A formulation as claimed in claim 11 wherein the insoluble hydrocolloid component comprises modified cellulose.

13. A formulation as claimed in claim 12 wherein the modified cellulose is microcrystalline cellulose.

14. A formulation as claimed in claim 1 wherein the total amount of the gelatinising stabiliser in the formulation is at least 0.2% w/v.

15. A formulation as claimed in claim 14 wherein the total amount of the gelatinising stabiliser in the formulation is 0.25 to 5% w/v.

16. A formulation as claimed in claim 9 wherein the gelatinising stabiliser comprises microcrystalline cellulose, guar gum and xanthan gum in a weight ratio of microcrystalline cellulose: guar gum: xanthan gum of 5:2-3:1.5-2.5.

17. A formulation as claimed in claim 1 comprising at least 8% w/v, based on the total weight of the formulation, of amino acid moieties.

18. A formulation as claimed in claim 17 comprising 8% to 55% w/v, based on the total weight of the formulation of amino acid moieties.

19. A formulation as claimed in claim 1 also comprising calcium and additional minerals.

20. A formulation as claimed in claim 1 comprising trace elements.

21. A formulation as claimed in claim 1 comprising vitamins.

22. A formulation as clamed in claim 1 comprising a fat.

23. A formulation as claimed in claim 22 wherein the fat comprises a blend of high oleic sunflower oil, canola oil and walnut oil.

24. A formulation as claimed in claim 23 wherein the fat is present in an amount up to 2% w/v.

25. A method of producing a formulation as claimed in claim 1 comprising preparing a slurry comprised of the ingredients of the formulation and effecting homogenisation of the slurry.

26. A method as claim in claim 25 wherein the homogenisation is a two stage homogenisation procedure comprising a first homogenisation step effected at a first pressure and a second homogenisation step effected at a second lower pressure.

27. A method as claimed in claim 26 wherein said first pressure is about 150-170 bar and the second pressure is about 30-50 bar.
